(19) United States
(12) Patent Application Publication
(10) Pub. No.: US 2007/0225284 A1
(43) Pub. Date: Sep. 27, 2007

(54) Inhibitors of soluble adenylate cyclase

(76) Inventors: Bernd Buchmann, Hohen Neuendorf (DE); Dirk Kosemund, Erfurt (DE); Bernd Menzenbach, Jena (DE); Martin Fritsch, Berlin (DE)

Correspondence Address: MILLEN, WHITE, ZELANO & BRANIGAN, P.C., 2200 CLARENDON BLVD., SUITE 1400, ARLINGTON, VA, 22201, US

(21) Appl. No.: 11/727,168

(22) Filed: Mar. 23, 2007

Related U.S. Application Data
(60) Provisional application No. 60/788,076, filed on Apr. 3, 2006.

(30) Foreign Application Priority Data
Mar. 23, 2006 (DE) 10 2006 014 320.5

(51) Int. Cl. A61K 31/5377; A61K 31/496; A61K 31/454; A61K 31/4439; A61K 31/405; C07D 413/02; C07D 405/02; C07D 403/02

(52) U.S. Cl. 514/232.8; 514/323; 514/337; 514/414; 514/419; 546/201; 548/465; 548/492; 544/144; 544/373; 514/254.09; 546/278.1

(57) ABSTRACT

The invention relates to compounds of general formula I as well as the production and use thereof as a medication.

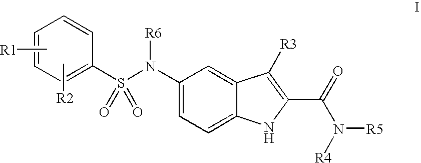

This application claims the benefit of the filing date of U.S. Provisional Application Ser. No. 60/788,076 filed Apr. 3, 2006.

This invention relates to inhibitors of soluble adenylate cyclase, its production as well as its use for the production of a pharmaceutical agent for contraception.

There are currently a number of modem contraceptive methods available for women; for male birth control, however, only very few methods are available (condom and sterilization). The development of new reliable agents for male birth control is absolutely necessary. In this connection, infertility produced by a “male pill” should be completely reversible and just as effective as the existing methods that are available to women. The infertility should set in relatively quickly and last as long as possible. Such contraceptive methods should not have any side effects; in addition to hormonal preparations, these can also be non-hormonal preparations in this connection. A possible starting point is the regulation of the activity of an enzyme, which plays an important role in the fertilization of an ovocyte, the soluble adenylate cyclase (sAC). This enzyme is expressed mainly in the testicular stem cells and is present in mature sperm.

In 1999, the authors Levin and Buck (Proc. Natl. Acad. Sci. USA 96 (1): 79-84) were able to purify and to clone an isoform of the sAC from the testes of rats.

The recombinant enzyme of rats can be stimulated by bicarbonate. By means of antibodies, it was possible to demonstrate that the catalytic domain of the enzyme is located in the testes, sperm, kidneys and the choroid plexus. These disclosures are the subject matter of the application WO01/85753, which was granted in the U.S. (U.S. Pat. No. 6,544,768).

In WO01/21829 (Conti et al.), isolated polynucleotide sequences that code for the human isoform of sAC, isolated sAC polypeptides and test systems are claimed with whose help substances can be identified that inhibit the activity of sAC. The possibility of using these substances to reduce the number of motile sperm cells in a reversible manner as well as their use as agents for male birth control are disclosed.

The John Herr group showed the isolation and characterization of the human isoform of sAC from sperm. In WO 02/20745, in addition to nucleic acids, the test systems that also code for sAC are claimed, with whose aid substances can be identified that modulate the expression or the activity of the human sAC. Such compounds could selectively inhibit, for example, the activity of sAC; this had the result that the sperm cells lose the ability to fertilize an ovocyte. These inhibitors of sAC therefore could be used as pharmaceutical agents for non-hormonal contraception.

The already known inhibitors of sAC indicate specific problems, however: catechol estrogens (T. Braun, Proc Soc Exp Biol Med 1990, 194(1): 58ff) and gossypol (K. L. Olgiati, Arch Biochem Biophys 1984, 231(2): 411ff) are inherently toxic, while adenosine analogs inhibit with only very weak action (M. A. Brown and E. R. Casillas, J Androl 1984, 5:361ff). The inhibitors (IC₅₀≦10 μmol) of the recombinant human sAC, which are described by Zippin et al. (J. H. Zippin et al. J Cell Biol 2004, 164(4): 527ff), are somewhat more potent.

To be able to make an agent for male birth control available, there is an increasing need for substances that are reversible, quick and successfully result in infertility.

This object is achieved by the provision of the compounds of general formula I 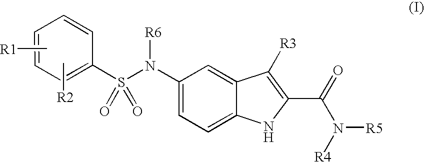
whereby
R¹ means hydrogen, halogen, CF₃, C₃-C₆-cycloalkyl, which optionally is polysaturated and optionally is polysubstituted, or the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl optionally can be interrupted in one or more places, in the same way or differently, by oxygen, sulfur or nitrogen, or the group sulfonyl-C₁-C₆-alkyl, sulfonamide, or cyano,
R² means halogen, CF₃, C₃-C₆-cycloalkyl, which optionally is polysaturated and optionally is polysubstituted, or the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl optionally can be interrupted in one or more places, in the same way or differently, by oxygen, sulfur or nitrogen, or the group sulfonyl-C₁-₆-alkyl, sulfonamide, or cyano,
R³ means C₆-C₁₂-aryl, which optionally can be substituted in one or more places, in the same way or differently, with halogen, with C₁-C₆-alkyl or C₁-C₆-acyl, which optionally can be substituted in one or more places, or can be substituted with C₁-C₆-alkoxy, hydroxy, cyano, CO₂-(C₁-C₆-alkyl), N-(C₁-C₆-alkyl)₂, CO—NR⁴R⁵ or with CF₃;
C₅-C₁₂-heteroaryl, which optionally can be substituted in one or more places, in the same way or differently, with halogen, C₁-C₆-alkyl, C₁-C₆-acyl, C₁-C₆-alkoxy, hydroxy, cyano, CO₂-(C₁-C₆-alkyl), N-(C₁-C₆-alkyl)₂, CO—NR⁴R⁵ or with CF₃; or
C₃-C₆-cycloalkyl, which optionally can be substituted in one or more places, in the same way or differently, with halogen, CF₃, hydroxy, cyano, CO₂-(C₁-C₆-alkyl), C₁-C₆-alkyl, C₁-C₆-acyl, N-(C₁-C₆-alkyl)₂, CO—NR⁴R⁵ or C₁-C₆-alkoxy,
R⁴ means hydrogen, C₃-C₆-cycloalkyl, which optionally is substituted in one or more places, in the same way or differently, with C₁-C₆-alkyl, C₁-C₆-acyl, C₁-C₆-alkoxy or CF₃;
C₆-C₁₂-aryl, which optionally is substituted in one or more places, in the same way or differently, with halogen, with C₁-C₆-alkyl, C₁-C₆-acyl, C₁-C₆-alkoxy, N-C₁-C₆-alkyl-C₁-C₆-alkyl, CF₃ or cyano; or
C₅-C₁₂-heteroaryl, which optionally is substituted in one or more places, in the same way or differently, with halogen, C₁-C₆-alkyl, C₁-C₆-acyl, C₁-C₆-alkoxy, N-C₁-C₆-alkyl-C₁-C₆-alkyl, CF₃ or cyano; or
C₁-C₆-alkyl, which can be substituted in any way desired,
R⁵ means hydrogen, C₁-C₆-alkyl-C₃-C₆-cycloalkyl, which optionally is substituted in one or more places, in the same way or differently, with C₁-C₆-alkyl, C₁-C₆-acyl, C₁-C₆-alkoxy or CF₃;
C₃-C₆-cycloalkyl, which optionally is substituted in one or more places, in the same way or differently, with C₁-C₆-alkyl, C₁-C₆-acyl, C₁-C₆-alkoxy or CF₃;
C₆-C₁₂-aryl, which optionally is substituted in one or more places, in the same way or differently, with halogen, C₁-C₆-alkyl, C₁-C₆-acyl, C₁-C₆-alkoxy, N-C₁-C₆-alkyl-C₁-C₆-alkyl, CF₃ or cyano; or
C₅-C₁₂-heteroaryl, which optionally is substituted in one or more places, in the same way or differently, with halogen, C₁-C₆-alkyl, C₁-C₆-acyl, C₁-C₆-alkoxy, N-C₁-C₆-alkyl-C₁-C₆-alkyl, CF₃ or cyano; or
C₁-C₆-alkyl, which can be substituted in any way desired, and
R⁴ and R⁵ together form a 5- to 8-membered ring, which can contain additional heteroatoms, and
R⁶ means the group C₁-C₆-alkyl, C₁-C₆-acyl, C₁-C₆-alkyl-cyclo-C₃-C₆-alkyl, or C₁-C₆-alkyl-C₆-C₁₂-aryl, in which C₁-C₆-alkyl, C₁-C₆-acyl, C₁-C₆-alkyl-cyclo-C₃-C₆-alkyl, or C₁-C₆-alkyl-C₆-C₁₂-aryl optionally can be substituted in one or more places, in the same way or differently, by hydroxy, methoxy, ethoxy, iso-propoxy, chlorine, bromine, fluorine, cyano, methyl-sulfonyl or amino-sulfonyl,
   as well as the isomers, diastereomers, enantiomers and salts thereof that overcome the known disadvantages and exhibit improved properties, i.e., good effectiveness, good solubility and stability.

The compounds according to the invention inhibit the soluble adenylate cyclase and thus prevent sperm capacitation and thus are used for male birth control.

Alkyl is defined in each case as a straight-chain or branched alkyl radical, such as, for example, methyl, ethyl, propyl, isopropyl, butyl, isobutyl, sec-butyl, tert-butyl, pentyl, isopentyl and hexyl.

Alkoxy is defined in each case as a straight-chain or branched alkoxy radical, such as, for example, methoxy, ethoxy, n-propoxy, iso-propoxy, n-butoxy, sec-butoxy, iso-butoxy, tert-butyloxy, pentoxy, iso-pentoxy and hexoxy.

Acyl is defined in each case as a straight-chain or branched radical, such as, for example, formyl, acetyl, propionyl, butyroyl, iso-butyroyl, valeroyl and benzoyl.

Cycloalkyls are defined as monocyclic alkyl rings such as cyclopropyl, cyclobutyl, cyclopentyl, and cyclohexyl.

Instead of the carbon atoms, the cycloalkyl radicals can contain one or more heteroatoms, such as oxygen, sulfur and/or nitrogen. Preferred are those heterocycloalkyls with 3 to 6 ring atoms. The ring systems, in which optionally one or more possible double bonds can be contained in the ring, are defined as, for example, cycloalkenyls, such as cyclopropenyl, cyclobutenyl, cyclopentenyl, cyclopentadienyl, cyclohexenyl, or cycloheptenyl, whereby the linkage both to the double bond and to the single bonds can be carried out.

Halogen is defined as fluorine, chlorine, bromine or iodine in each case.

In each case, the aryl radical comprises 6-12 carbon atoms and can be, for example, benzocondensed. For example, the following can be mentioned: phenyl, tropyl, cyclooctadienyl, indenyl, naphthyl, biphenyl, florenyl, anthracenyl etc.

The heteroaryl radical comprises 5-16 ring atoms in each case and instead of the carbon can contain in the ring one or more heteroatoms that are the same or different, such as oxygen, sulfur or nitrogen, and can be monocyclic, bicyclic or tricyclic, and in addition can be benzocondensed in each case.

For example, there can be mentioned:

Thienyl, furanyl, pyrrolyl, oxazolyl, imidazolyl, pyrazolyl, isoxazolyl, isothiazolyl, oxadiazolyl, triazolyl, thiadiazolyl, etc., and benzo derivatives thereof, such as, e.g., benzofuranyl, benzothienyl, benzooxazolyl, benzimidazolyl, indazolyl, indolyl, isoindolyl, etc.; or pyridazinyl, pyrimidinyl, pyrazinyl, triazinyl, etc., and benzo derivatives thereof, such as, e.g., quinolyl, isoquinolyl, etc.; or azocinyl, indolizinyl, purinyl, etc., and benzo derivatives thereof; or quinolinyl, isoquinolinyl, cinnolinyl, phthalazinyl, quinazolinyl, quinoxalinyl, naphthyridinyl, pteridinyl, carbazolyl, acridinyl, phenazinyl, phenothiazinyl, phenoxazinyl, xanthenyl, oxepinyl, etc.

The heteroaryl radical can be benzocondensed in each case. For example, thiophene, furan, oxazole, thiazole, imidazole, pyrazole and benzo derivatives thereof can be mentioned as 5-ring heteroaromatic compounds, and pyridine, pyrimidine, triazine, quinoline, isoquinoline and benzo derivatives can be mentioned as 6-ring heteroaromatic compounds.

Heteroatoms are defined as oxygen, nitrogen or sulfur atoms.

If an acid group is included, the physiologically compatible salts of organic and inorganic bases, such as, for example, the readily soluble alkali and alkaline-earth salts, as well as N-methyl-glucamine, dimethyl-glucamine, ethyl-glucamine, lysine, 1,6-hexadiamine, ethanolamine, glucosamine, sarcosine, serinol, tris-hydroxy-methyl-amino-methane, aminopropanediol, Sovak base, and 1-amino-2,3,4-butanetriol, are suitable as salts.

If a basic group is included, the physiologically compatible salts of organic and inorganic acids, such as hydrochloric acid, sulfuric acid, phosphoric acid, citric acid, tartaric acid, i.a., are suitable.

Those compounds of general formula (I), whereby
R¹ means hydrogen, halogen, CF₃, C₃-C₆-cycloalkyl, or the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl optionally can be interrupted in one or more places, in the same way or differently, by oxygen, sulfur or nitrogen, or the group sulfonyl-C₁-C₆-alkyl, sulfonamide, or cyano,
R² means halogen, CF₃, or C₃-C₆-cycloalkyl, or the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl optionally can be interrupted in one or more places, in the same way or differently, by oxygen, sulfur or nitrogen, or the group sulfonyl-C₁-C₆-alkyl, sulfonamide, or cyano,
R³ means C₆-C₁₂-aryl, which optionally can be substituted in one or more places, in the same way or differently, with halogen, C₁-C₆-alkyl, C₁-C₃-acyl, C₁-C₃-alkoxy, cyano, hydroxy, N—(CH₃)₂, CO₂-(C₁-C₃-alkyl), CO—NR⁴R⁵ or CF₃;
C₅-C₁₂-heteroaryl, which optionally can be substituted in one or more places, in the same way or differently, with chlorine and/or fluorine, with C₁-C₆-alkyl, C₁-C₃-acyl, C₁-C₃-alkoxy, cyano, hydroxy, N—(CH₃)₂, CO₂-(C₁-C₃-alkyl), CO—NR⁴R⁵ or with CF₃;
C₃-C₆-cycloalkyl, which optionally can be substituted in one or more places, in the same way or differently, with chlorine and/or fluorine, CF₃, cyano, C₁-C₃-alkyl, C₁-C₃-acyl, hydroxy, N—(CH₃)₂, CO₂-(C₁-C₃-alkyl), CO—NR⁴R⁵ or C₁-C₃-alkoxy,
R⁴ means hydrogen, C₃-C₆-cycloalkyl, which optionally is substituted in one or more places, in the same way or differently, with C₁-C₃-alkyl, C₁-C₃-acyl, C₁-C₃-alkoxy or CF₃;
C₆-C₁₂-aryl, which optionally is substituted in one or more places, in the same way or differently, with halogen, C₁-C₃-alkyl, C₁-C₃-acyl, C₁-C₃-alkoxy, N-C₁-C₃-alkyl-C₁-C₃-alkyl, CF₃ or cyano; or
C₅-C₁₂-heteroaryl, which optionally is substituted in one or more places, in the same way or differently, with halogen, with C₁-C₃-alkyl, C₁-C₃-acyl, C₁-C₃-alkoxy, N-C₁-C₃-alkyl-C₁-C₃-alkyl, CF₃ or cyano; or
C₁-C₆-alkyl, which can be substituted in any way desired,
R⁵ means hydrogen, C₁-C₆-alkyl-C₃-C₆-cycloalkyl, which optionally is substituted in one or more places, in the same way or differently, with C₁-C₆-alkyl, C₁-C₆-acyl, C₁-C₆-alkoxy or CF₃;
C₃-C₆-cycloalkyl, which optionally is substituted in one or more places, in the same way or differently, with C₁-C₃-alkyl, C₁-C₃-acyl, C₁-C₃-alkoxy or CF₃;
C₆-C₁₂-aryl, which optionally is substituted in one or more places, in the same way or differently, with halogen, C₁-C₃-alkyl, C₁-C₃-acyl, C₁-C₃-alkoxy, N-C₁-C₃-alkyl-C₁-C₃-alkyl, CF₃ or cyano; or
C₅-C₁₂-heteroaryl, which optionally is substituted in one or more places, in the same way or differently, with halogen, with C₁-C₃-alkyl, C₁-C₃-acyl, C₁-C₃-alkoxy, N-C₁-C₃-alkyl-C₁-C₃-alkyl, CF₃ or cyano; or
C₁-C₆-alkyl, which can be substituted in any way desired,
R⁴ and R⁵ together form a 5- to 8-membered ring, which can contain additional heteroatoms, and
R⁶ means the group C₁-C₆-alkyl, C₁-C₆-alkyl-cyclo-C₃-C₆-alkyl, or C₁-C₆-alkyl-C₆-C₁₂-aryl, in which C₁-C₆-alkyl, C₁-C₆-alkyl-cyclo-C₃-C₆-alkyl, or C₁-C₆-alkyl-C₆-C₁₂-aryl optionally can be substituted in one or three places, in the same way or differently, by hydroxy, methoxy, ethoxy, iso-propoxy, chlorine, bromine, fluorine, cyano, methyl-sulfonyl or amino-sulfonyl,
   as well as the isomers, diastereomers, enantiomers and salts thereof, are especially preferred.

Those compounds of general formula I, whereby
R¹ means hydrogen,
R² means C₃-C₆-cycloalkyl, C₁-C₆-alkyl, CF₃, cyano, bromine, or the group —OCF₃, or —SO₂—CH₃,
R³ means C₆-C₁₂-aryl, which optionally can be substituted in one or more places, in the same way or differently, with halogen, C₁-C₆-alkyl, C₁-C₃-acyl, C₁-C₃-alkoxy, cyano, hydroxy, N—(CH₃)₂, CO₂-(C₁-C₃-alkyl), CO—NR⁴R⁵ or CF₃;
C₅-C₁₂-heteroaryl, which optionally can be substituted in one or more places, in the same way or differently, with chlorine and/or fluorine, with C₁-C₆-alkyl, C₁-C₃-acyl, C₁-C₃-alkoxy, cyano, hydroxy, N—(CH₃)₂, CO₂-(C₁-C₃-alkyl), CO—NR⁴R⁵ or with CF₃;
C₃-C₆-cycloalkyl, which optionally can be substituted in one or more places, in the same way or differently, with chlorine and/or fluorine, CF₃, cyano, C₁-C₃-alkyl, C₁-C₃-acyl, hydroxy, N—(CH₃)₂, CO₂-(C₁-C₃-alkyl), CO—NR⁴R⁵ or C₁-C₃-alkoxy,
R⁴ means hydrogen,
R⁵ means hydrogen, C₁-C₆-alkyl-C₃-C₆-cycloalkyl, which optionally is substituted in one or more places, in the same way or differently, with C₁-C₆-alkyl, C₁-C₆-acyl, C₁-C₆-alkoxy or CF₃;
C₃-C₆-cycloalkyl, which optionally is substituted in one or more places, in the same way or differently, with C₁-C₃-alkyl, C₁-C₃-acyl, C₁-C₃-alkoxy or CF₃;
C₆-C₁₂-aryl, which optionally is substituted in one or more places, in the same way or differently, with halogen, C₁-C₃-alkyl, C₁-C₃-acyl, C₁-C₃-alkoxy, N-C₁-C₃-alkyl-C₁-C₃-alkyl, CF₃ or cyano; or
C₅-C₁₂-heteroaryl, which optionally is substituted in one or more places, in the same way or differently, with halogen, C₁-C₃-alkyl, C₁-C₃-acyl, C₁-C₃-alkoxy, N-C₁-C₃-alkyl-C₁-C₃-alkyl, CF₃ or cyano; or C₁-C₆-alkyl, which can be substituted in any way desired,
R⁶ means the group C₁-C₄-alkyl, CH₂-cyclo-C₃-C₆-alkyl, or CH₂-C₆-C₁₂-aryl, in which C₁-C₄-alkyl, CH₂-cyclo-C₃-C₆-alkyl, or CH₂-C₆-C₁₂-aryl optionally can be substituted in one or three places, in the same way or differently, by hydroxy, methoxy, chlorine, fluorine, cyano or amino-sulfonyl,
   as well as the isomers, diastereomers, enantiomers and salts thereof, are also preferred.

Those compounds of general formula I, whereby
R¹ means hydrogen,
R² means C₃-C₆-cycloalkyl, C₁-C₆-alkyl, CF₃, cyano, bromine, or the group —OCF₃, or —SO₂—CH₃ and is in para-position,
R³ means C₆-C₁₂-aryl, which optionally can be substituted in one or two places, in the same way or differently, with halogen, C₁-C₃-alkyl, acetyl, methoxy, ethoxy, cyano, hydroxy, N—(CH₃)₂, CO₂-(C₁-C₃-alkyl), CO—NHR⁵ or CF₃;
C₅-C₁₂-heteroaryl, which optionally can be substituted in one or two places, in the same way or differently, with chlorine and/or fluorine, with C₁-C₃-alkyl, acetyl, methoxy, ethoxy, cyano, hydroxy, N(CH₃)₂, CO₂-(C₁-C₃-alkyl), CO—NHR⁵ or with CF₃; or C₃-C₆-cycloalkyl,
R⁴ means hydrogen,
R⁵ means hydrogen, or C₁-C₆-alkyl-C₃-C₆-cycloalkyl, which optionally is substituted in one or more places, in the same way or differently, with C₁-C₆-alkyl, C₁-C₆-acyl, C₁-C₆-alkoxy or CF₃;
C₃-C₆-cycloalkyl, which optionally is substituted in one or more places, in the same way or differently, with C₁-C₃-alkyl, C₁-C₃-acyl, C₁-C₃-alkoxy or CF₃;
C₆-C₁₂-aryl, which optionally is substituted in one or more places, in the same way or differently, with halogen, C₁-C₃-alkyl, C₁-C₃-acyl, C₁-C₃-alkoxy, N-C₁-C₃-alkyl-C₁-C₃-alkyl, CF₃ or cyano; or
C₅-C₁₂-heteroaryl, which optionally is substituted in one or more places, in the same way or differently, with halogen, C₁-C₃-alkyl, C₁-C₃-acyl, C₁-C₃-alkoxy, N-C₁-C₃-alkyl-C₁-C₃-alkyl, CF₃ or cyano; or
C₁-C₆-alkyl, which can be substituted in any way desired,
R⁶ means the group C₁-C₄-alkyl, CH₂-cyclo-C₃-C₆-alkyl, or CH₂-C₆-C₁₂-aryl, in which C₁-C₄-alkyl, CH₂-cyclo-C₃-C₆-alkyl, or CH₂-C₆-C₁₂-aryl optionally can be substituted in one or three places, in the same way or differently, by hydroxy, methoxy, chlorine, fluorine, cyano or amino-sulfonyl,
   as well as the isomers, diastereomers, enantiomers and salts thereof, are also preferred.

Those compounds of general formula I, whereby
R¹ means hydrogen,
R² means tert-butyl, iso-propyl, iso-butyl, sec-butyl, cyano, bromine, or the group —O—CF₃, or —SO₂—CH₃ and is in para-position,
R³ means the group 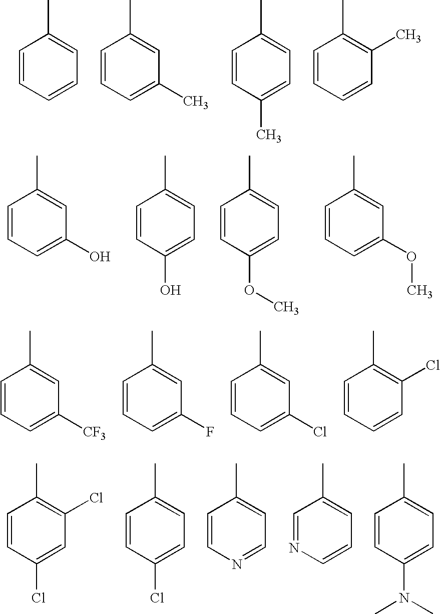
R⁴ means hydrogen,
R⁵ means hydrogen or the group —(CH₂)ₙ—N—(CH₃)₂, —(CH₂)₂—CH₃, —CH₂)₂—NH—COCH₃, —(CH₂)—CHCH₃—OH, —CH₂)₂—O—CH₃, —(CH₂)₂—OH, or —CHCH₃—CH₂—OH, whereby m=1-3, 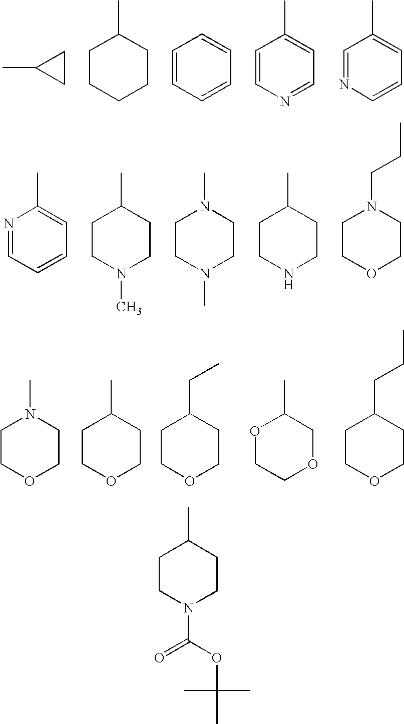
R⁶ means methyl, ethyl, propyl, 2-methoxy-ethyl, —CH₂—CF₃, —(CH₂)₂—CF₃ and benzyl,
   as well as the isomers, diastereomers, enantiomers and salts thereof, are also preferred.

Those compounds of general formula I, in which
R¹ means hydrogen,
R² means tert-butyl, iso-propyl, iso-butyl, sec-butyl, cyano, bromine, or the group —O—CF₃, or —SO₂—CH₃ and is in para-position,

R³ means the group

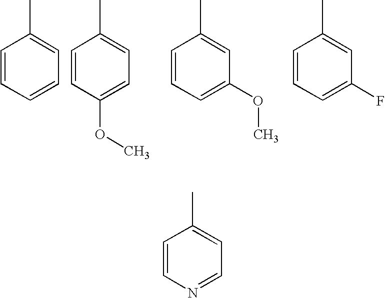

R⁴ means hydrogen,

R⁵ means hydrogen or the group —CH₂)—CHCH₃—OH, —(CH₂)₂—O—CH₃, or —CHCH₃—CH₂—OH,

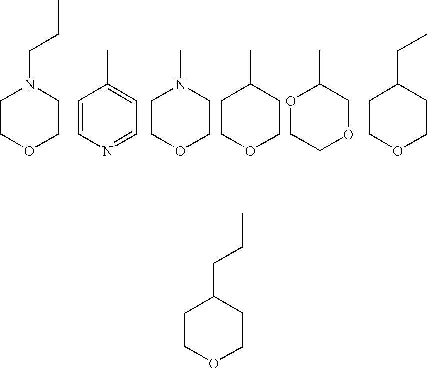

R⁶ means methyl, ethyl, propyl, 2-methoxy-ethyl, —CH₂—CF₃, —(CH₂)₂—CF₃ and benzyl,

as well as the isomers, diastereomers, enantiomers and salts thereof, are also preferred.

The following compounds corresponding to this invention are quite especially preferred:

1.5-[(4-tert-Butyl-phenylsulfonyl)-methyl-amino]-3-phenyl-1H-indole-2-carboxylic acid-(tetrahydro-pyran-4-yl)-amide

2. 5-[(4-tert-Butyl-phenylsulfonyl)-methyl-amino]-3-phenyl-1H-indole-2-carboxylic acid (2-morpholin-4-yl-ethyl)-amide

3. (±)-5-[(4-tert-Butyl-phenylsulfonyl)-methyl-amino]-3-phenyl-1H-indole-2-carboxylic acid-(2-hydroxy-1-methyl-ethyl)-amide

4. (±)-5-[(4-tert-Butyl-phenylsulfonyl)-methyl-amino]-3-phenyl-1H-indole-2-carboxylic acid-(2-hydroxy-propyl)-amide

5. 5-[(4-tert-Butyl-phenylsulfonyl)-methyl-amino]-3-phenyl-1H-indole-2-carboxylic acid-(pyridin-4-yl)-amide

6. 5-[(4-tert-Butyl-phenylsulfonyl)-benzyl-amino]-3-phenyl-1H-indole-2-carboxylic acid-(tetrahydro-pyran-4-yl)-amide

7. 5-[(4-tert-Butyl-phenylsulfonyl)-benzyl-amino]-3-phenyl-1H-indole-2-carboxylic acid (2-morpholin-4-yl-ethyl)-amide

8. (±)-5-[(4-tert-Butyl-phenylsulfonyl)-benzyl-amino]-3-phenyl-1H-indole-2-carboxylic acid-(2-hydroxy-1-methyl-ethyl)-amide

9. (±)-5-[(4-tert-Butyl-phenylsulfonyl)-benzyl-amino]-3-phenyl-1H-indole-2-carboxylic acid-(2-hydroxy-propyl)-amide

10. 5-[(4-tert-Butyl-phenylsulfonyl)-(2-methoxy-ethyl)-amino]-3-phenyl-1H-indole-2-carboxylic acid-(tetrahydro-pyran-4-yl)-amide

11. 5-[(4-tert-Butyl-phenylsulfonyl)-(2-methoxy-ethyl)-amino]-3-phenyl-1H-indole-2-carboxylic acid (2-morpholin-4-yl-ethyl)-amide

12. (±)-5-[(4-tert-Butyl-phenylsulfonyl)-(2-methoxy-ethyl)-amino]-3-phenyl-1H-indole-2-carboxylic acid-(2-hydroxy-1-methyl-ethyl)-amide

13. (±)-5-[(4-tert-Butyl-phenylsulfonyl)-(2-methoxy-ethyl)-amino]-3-phenyl-1H-indole-2-carboxylic acid-(2-hydroxy-propyl)-amide

14. 5-[(4-tert-Butyl-phenylsulfonyl)-methyl-amino]-3-(3-fluoro-phenyl)-1H-indole-2-carboxylic acid-(tetrahydro-pyran-4-yl)-amide

15. 5-[(4-tert-Butyl-phenylsulfonyl)-methyl-amino]-3-(3-fluoro-phenyl)-1H-indole-2-carboxylic acid (2-morpholin-4-yl-ethyl)-amide

16. 5-[(4-tert-Butyl-phenylsulfonyl)-methyl-amino]-3-(3-methoxy-phenyl)-1H-indole-2-carboxylic acid-(tetrahydro-pyran-4-yl)-amide

17. 5-[(4-tert-Butyl-phenylsulfonyl)-methyl-amino]-3-(3-methoxy-phenyl)1H-indole-2-carboxylic acid (2-morpholin-4-yl-ethyl)-amide 18.

In addition, the invention relates to a process for the production of the compounds of general formula I according to the invention, which is characterized in that a compound of formula II 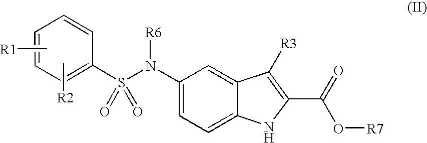
in which R¹, R², R³, and R⁶ have the above-indicated meanings and R⁷ can be a hydrogen or a C₁-C₆-alkyl radical, and is preferably hydrogen, the methyl or ethyl radical, is reacted with an amine of general formula III 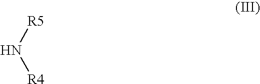
in which R⁴ and R⁵ have the above-indicated meaning, according to the methods that are known to one skilled in the art, and optionally required protective groups are then cleaved.

For the case that R⁷ is equal to hydrogen, the reaction first can be carried out by activating the acid function; in this case, for example, first the carboxylic acid of general formula II is converted into the mixed anhydride in the presence of a tertiary amine, such as, for example, triethylamine, with isobutyl chloroformate. The reaction of the mixed anhydride with the alkali salt of the corresponding amine is carried out in an inert solvent or solvent mixture, such as, for example, tetrahydrofuran, dimethoxyethane, dimethylformamide, or hexamethylphosphoric acid triamide, at temperatures of between −30° C. and +60° C., preferably at 0° C. to 30° C.

Another possibility consists in activating the carboxylic acid of general formula II by reagents, such as, for example, HOBt or HATU. The reaction of the acid is carried out with, e.g., HATU in an inert solvent, such as, for example, DMF in the presence of the corresponding amine of general formula III and a tertiary amine, such as, for example, ethyldiisopropylamine at temperatures of between −50 and +60° C., preferably at 0° C. to 30° C.

For the case that R⁶ is equal to C₁-C₆-alkyl, for example, a direct amidolysis of the ester with the corresponding amine optionally also can be performed with the aid of aluminum trialkyl reagents, preferably aluminum trimethyl.

The compounds of general formula II that are used as starting materials can be produced, for example, in which in a way that is known in the art, the nitro group in the known indole esters IV 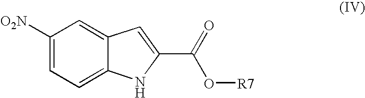
in which R⁷ is a C₁-C₆-alkyl radical, preferably a methyl or ethyl radical, is reduced in a hydrogen atmosphere or a hydrogen source, such as, for example, ammonium formate, in the presence of a Pd catalyst first to the amino function, and then this amine is reacted with a halide of general formula V 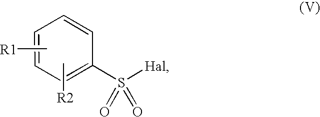
in which R¹ and R² have the above-indicated meanings and Hal stands for a halogen, preferably fluoride, chloride or bromide, in the presence of a base, such as, for example, pyridine, diisopropylethylamine, triethylamine or potassium carbonate, to form the compounds of general formula VI

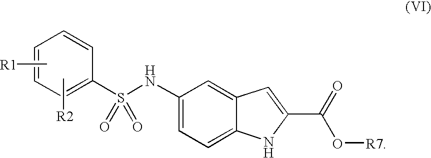

The esters of general formula VI are then halogenated in the 3-position, for example, by means of iodine, NBI, NBS or else CuBr₂, and thus the compounds of general formula VII are obtained 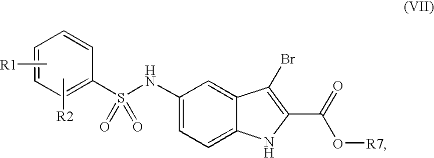
in which R¹, R² and R⁷ have the above-indicated meanings.

Then, these esters are reacted in the presence of a base, such as, for example, diisopropylethylamine, potassium carbonate or cesium carbonate in acetone or tetrahydrofuran with the halide of general formula VIII 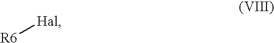
in which R6 has the above-indicated meaning, and Hal stands for a halogen, preferably iodide, chloride, or bromide, to form the compounds of general formula IX

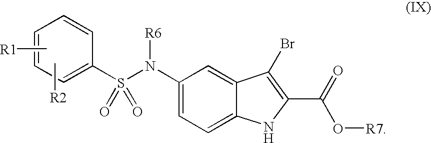

The esters of general formula IX are then converted in the 3-position in a Pd-catalyzed reaction with boronic acid derivatives of general formula X 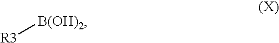
in which R³ has the above-indicated meaning, optionally after cleavage of required protective groups in R⁶, optionally followed by a saponification, for example with sodium hydroxide solution, into the compounds of general formula II

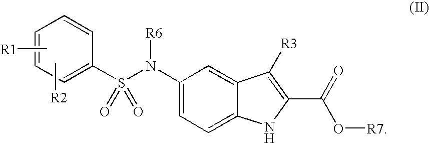

As an alternative, the possibility existed to convert the esters of general formula VII first in the 3-position in a Pd-catalyzed reaction with the boronic acid derivatives of general formula X 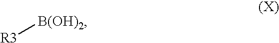
in which R³ has the above-indicated meaning, into the compounds of general formula XI 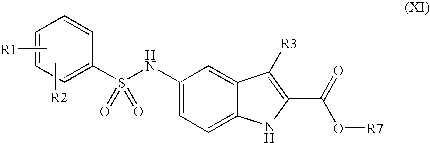
in which R¹, R², R³ and R⁷ have the above-indicated meanings, and then the N-alkylation step is performed in the presence of a base, such as, for example, diisopropylethylamine, potassium carbonate or cesium carbonate in acetone or tetrahydrofuran and the halide of general formula VIII 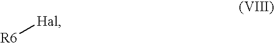
in which R⁶ has the above-indicated meaning, and Hal stands for a halogen, preferably iodide, chloride or bromide, optionally after cleavage of the required protective groups in R⁶, optionally followed by a saponification with, for example, sodium hydroxide solution, to form the compounds of general formula II.

The compounds according to the invention inhibit the soluble adenylate cyclase, upon which their action is based, for example, in the case of male birth control.

Adenylate cyclases are the effector molecules for one of the most used signal transduction methods; they synthesize the second messenger molecule of cyclic adenosine monophosphate (cAMP) from adenosine triphosphate (ATP) with cleavage of pyrophosphate (PP). cAMP mediates numerous cellular responses for a number of neurotransmitters and hormones. The soluble, sperm-specific adenylate cyclase (sAC, human mRNA sequence (gene bank) NM_{—}018417, human gene ADCY X) is one of ten described adenylate cyclases in the human genome. In this case, sAC shows several specific properties that are distinguished from the other adenylate cyclases. In contrast to all other adenylate cyclases, sAC is stimulated by the concentration of bicarbonate in the medium that surrounds it and not by G-proteins. sAC does not have any transmembrane regions in its amino acid sequence; it cannot be inhibited by forskolin, can be stimulated much more strongly by manganese than by magnesium, and shows only low sequence homologies to the other adenylate cyclases (≦26% identity of the catalytic domains I and II of sAC with other adenylate cyclases on the amino acid plane).

Specific, manganese-dependent activity of sAC was first described by T. Braun et al. (1975, PNAS 73:1097ff) in rat testes and sperm. N. Okamura et al. (1985, J. Biol. Chem 260(17): 9699ff) showed that the substance that stimulates the activity of sAC in the pig seminal fluid is bicarbonate. It could also be shown that only in the rat testis and sperm, but not in other tissues, AC activity that can be stimulated by bicarbonate can be detected. sAC was purified from the rat testis by the Buck and Levin group and sequenced for the first time (J. Buck et al. 1999 PNAS 96:79ff, WO 01/85753). The properties that are to be expected (e.g., the ability to stimulate bicarbonate and magnesium) were confirmed in the recombinantly-expressed protein (Y. Chen et al. 2000 Science 289:625ff).

Data regarding the distribution of sAC mRNA and the sAC activity that can be stimulated by bicarbonate can indicate a testis- and sperm-specific expression of the enzyme (M. L. Sinclair et al. 2000 Mol Reprod Develop 56:6ff; N. Okamura et al. 1985, J. Biol. Chem 260(17):9699 ff; J. Buck et al. 1999 PNAS 96:79ff). In this case, in the testicles, sAC mRNA is expressed only in later stages of the gametes that develop into sperm, but not in somatic cells (M. L. Sinclair et al. 2000 Mol Reprod Develop 56:6ff).

Regarding the function of sAC in sperm in mammals, there are a number of pharmacological studies. Before sperm penetrate the zona pellucida of the egg, so as to subsequently merge with the oolemma of the egg, they must be prepared for this functionality. This process, the sperm capacitation, is quite well studied. A capacitated sperm is distinguished by an altered pattern of movement and by the ability to go through the process of acrosomal reaction by a suitable stimulus (a release of lytic enzymes that are presumably used in the penetration of the zona pellucida by the sperm). The sperm capacitation is carried out in vivo and in vitro, i.a., based on an elevated bicarbonate concentration in the medium (P. E. Visconti & G. S. Kopf (1998) Biol Reprod 59:1ff; E. de Lamirande et al. 1997 Mol Hum Reprod 3(3):175ff). The sperm capacitation can also be stimulated by the addition of suitable membrane-penetrating cAMP analogs, e.g., db-cAMP and an inhibitor that inhibits their degradation (e.g., IBMX). The expected dependence of the sperm function of sAC was confirmed only recently by a genetic deletion model, a so-called knock-out mouse (G. Esposito et al. 2004 PNAS 101(9):2993ff). Male mice in which the gene for sAC is lacking show a normal spermatogenesis but are infertile. The sperm have motility defects and are not able to fertilize an egg. The animals showed no other defects or abnormal findings, which corresponds to other hypothesized functions of the sAC (J. H. Zippin et al 2003 FASEB 17:82ff)).

The sAC has a unique sequence and only a slight homology to other somatic adenylate cyclases. It is the sole adenylate cyclase in mammal sperm, and the activity is essential to the mobility of the sperm and the capacitation. Specific inhibitors of sAC accordingly represent an important possibility of regulating male fertility.

Pharmaceutical agents that contain at least one of the compounds according to claims 1-7 are therefore subjects of this invention.

The use of the compounds according to claims 1-7 is also a subject of this invention.

To use the compounds according to the invention as pharmaceutical agents, the latter are brought into the form of a pharmaceutical preparation that in addition to the active ingredient for enteral or parenteral administration contains suitable pharmaceutical, organic or inorganic inert carrier materials, such as, for example, water, gelatin, gum arabic, lactose, starch, magnesium stearate, talc, vegetable oils, polylalkylene glycols, etc. The pharmaceutical preparations can be present in solid form, for example as tablets, coated tablets, suppositories, or capsules, or in liquid form, for example as solutions, suspensions, or emulsions. Moreover, they optionally contain adjuvants, such as preservatives, stabilizers, wetting agents or emulsifiers, salts for altering the osmotic pressure, or buffers. These pharmaceutical preparations are also subjects of this invention.

For parenteral administration, in particular injection solutions or suspensions, in particular aqueous solutions of active compounds in polyhydroxyethoxylated castor oil, are suitable.

As carrier systems, surface-active adjuvants, such as salts of bile acids or animal or plant phospholipids, but also mixtures thereof as well as liposomes or their components can also be used.

For oral administration, in particular tablets, coated tablets or capsules with talc and/or hydrocarbon vehicles or binders, such as, for example, lactose, corn or potato starch, are suitable. The administration can also be carried out in liquid form, such as, for example, as a juice, to which optionally a sweetener is added.

For the vaginal application, e.g., suppositories are suitable and common.

The enteral, parenteral and oral administrations are also subjects of this invention.

The dosage of the active ingredients can vary depending on the method of administration, age and weight of the patient, type and severity of the disease to be treated and similar factors. The daily dose is 0.5-1000 mg, preferably 50-200 mg, whereby the dose can be given as a single dose that is to be administered once or subdivided into 2 or more daily doses.

The compounds of general formula I according to the invention are, i.a., excellent inhibitors of the soluble adenylate cyclase. Inhibitors of the soluble adenylate cyclase lead to a reduction of the cAMP signal. The cAMP level is decisive for the monitoring of the processes that play an important role in cell proliferation, cell differentiation and apoptosis. Diseases, such as, e.g., cancer, in which the reduction of the cAMP level is decisive, can be modulated by inhibitors of soluble adenylate cyclase. This modulation can have prophylactic and therapeutic effects for the patients that suffer from such a disease. Diseases that, like cancer, are accompanied by an elevated cell proliferation are currently treated by, e.g., radiation therapy and chemotherapy. These processes are non-specific and have a high potential for side effects. The preparation of new substances that directly attack specific target sites is therefore advantageous. Substances that modulate the cAMP production by the inhibition of soluble adenylate cyclases are subjects of this invention. Thus, for example, the anomal cell proliferation can be reduced or inhibited by regulation or inhibition of the cAMP production. By the use of the substances according to the invention, the soluble adenylate cyclase can be inhibited; this has the result of a reduction of the cell proliferation. Subjects of this invention are pharmaceutical agents for treating diseases that contain at least one compound according to general formula I, as well as pharmaceutical agents with suitable formulation substances and vehicles. The diseases are thus characterized in that they are caused by disorders of the metabolism of the second messenger cAMP.

A reduction of the cAMP concentration by inhibition of the soluble adenylate cyclase can make available agents for modulation of the sperm capacitation. A subject of this invention is the use of the substances according to the invention for reduction and/or inhibition of male gamete fertility mediated by the reduction or inhibition of soluble adenylate cyclase activity and the thus resulting sperm capacitation.

The fertilization of the ovum can be prevented by the administration of an effective amount of a substance that results in the inhibition of the cAMP production. The use of the compound of general formula I for the production of a pharmaceutical agent for non-hormonal contraception is also a subject of this invention.

If the production of the starting compounds is not described, the latter are known or can be produced analogously to known compounds or to processes that are described here. It is also possible to implement all reactions that are described here in parallel reactors or by means of combinatory operating procedures.

The isomer mixtures can be separated into enantiomers or E/Z isomers according to commonly used methods, such as, for example, crystallization, chromatography or salt formation.

The production of salts is carried out in the usual way by a solution of the compound of formula I being mixed with the equivalent amount of or an excess of a base or acid, which optionally is in solution, and the precipitate being separated or the solution being worked up in the usual way.

Production of the Compounds According to the Invention

The examples below explain the production of the compounds of general formula (I) according to the invention, without limiting the scope of the claimed compounds to these examples.

The compounds of general formula (I) according to the invention can be produced as described below.

### EXAMPLE 1

### 5-[(4-tert-Butyl-phenylsulfonyl)-methyl-amino)]-3-phenyl-1H-indole-2-carboxylic acid-(tetrahydro-pyran-4-yl)-amide

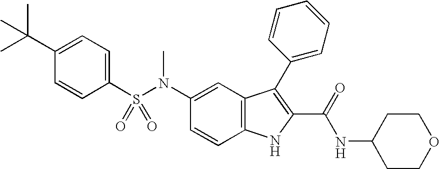

40.3 mg of N-[(dimethylamino)-1H-1,2,3-triazolo[4,5-b]pyridin-1-ylmethylene]-N-methylmethanaminiumhexafluorophosphate-N-oxide (HATU) and 9.84 mg of 4-aminotetrahydropyran are added to a solution of 45 mg of the acid, produced in Example 1f), in 0.75 ml of dimethylformamide. At 0° C., 18.0 μl of ethyldiisopropylamine is then added in drops and stirred for 20 hours at room temperature. Then, 25 ml of water is added, stirred for 30 minutes and suctioned off. The thus obtained residue is purified by chromatography on silica gel with hexane/0-70% ethyl acetate. In this way, 49.7 mg of the title compound is obtained.

NMR (300 MHz, DMSO-d6): δ=1.22 (2H), 1.26 (s, 9H), 1.65 (2H), 3.07 (3H), 3.32 (2H), 3.68 (2H), 3.89 (1H), 6.91 (1H), 6.99 (1H), 7.26-7.33 (4H), 7.34-7.41 (5H), 7.54 (2H), 11.85 (1H).

The starting material for the above-mentioned title compound is produced as follows:

1a) 5-Amino-1H-indole-2-carboxylic acid ethyl ester

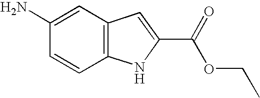

5 g of 5-nitro-1H-indole-2-carboxylic acid ethyl ester is introduced into 170 ml of methanol and 0.5 ml of water, mixed with 6.73 g of ammonium formate and with 50 mg of palladium on carbon (10%) and refluxed for 1 hour at 90° C. Then, it is suctioned off on Celite and rewashed with warm methanol. After the solvent is removed, the residue is mixed with 100 ml of water, stirred for 10 minutes, and the precipitated solid is suctioned off on a G4 frit. The thus obtained solid is dried in a vacuum. In this way, 4.12 g of the title compound is obtained.

NMR (300 MHz, DMSO-d6): δ=1.28 (3H), 4.25 (2H), 4.63 (2H), 6.62-6.68 (2H), 6.79 (1H), 7.12 (1H), 11.35 (1H).

1b) 5-(4-tert-Butyl-phenylsulfonylamino)-1H-indole-2-carboxylic acid ethyl ester

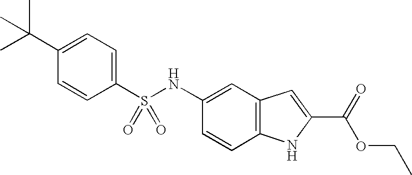

5.18 ml of ethyldiisopropylamine and 4.69 g of 4-tert-butyl-phenylsulfonyl chloride are added to a solution of 4.12 g of the amine, produced in Example 1a), in 195 ml of DMF at 0° C., and it is stirred for two hours at room temperature. The solvent is removed under reduced pressure, and the residue is purified by chromatography on silica gel with hexane/0-80% ethyl acetate. In this way, 7.56 g of the title compound is obtained.

NMR (300 MHz, DMSO-d6): δ=1.20 (9H), 1.27 (3H), 4.27 (2H), 6.97-7.03 (2H), 7.25 (1H), 7.31 (1H), 7.48 (2H), 7.59 (2H), 9.93 (1H), 11.80 (1H).

1c) 3-Bromo-5-(4-tert-butyl-phenylsulfonylamino)-1H-indole-2-carboxylic acid ethyl ester

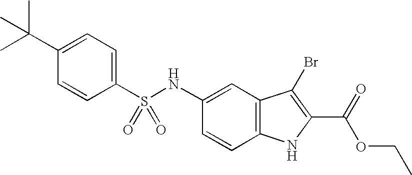

3.36 g of N-bromosuccinimide is added to a solution of 7.56 g of the sulfonamide, produced in Example 1b), in 217 ml of tetrahydrofuran, and it is stirred for 40 minutes at room temperature. It is diluted with 300 ml of ethyl acetate, washed once with 50 ml of water, twice with 50 ml each of saturated sodium chloride solution, and the organic phase is dried on sodium sulfate. After filtration and concentration by evaporation in a vacuum, the thus obtained residue is recrystallized from hexane/ethyl acetate. In this way, 8.11 g of the title compound is obtained.

NMR (300 MHz, DMSO-d6): δ=1.20 (9H), 1.30 (3H), 4.31 (2H), 7.08-7.15 (2H), 7.33 (1H), 7.50 (2H), 7.60 (2H), 10.08 (1H), 12.16 (1H).

1d) 3-Bromo-5-[(4-tert-butyl-phenylsulfonyl)-methyl-amino]-1H-indole-2-carboxylic acid ethyl ester

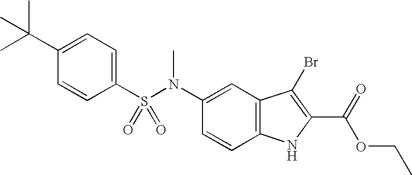

375 mg of potassium carbonate and 0.13 ml of methyl iodide are added to a suspension of 1 g of the bromide, produced in Example 1c), in 10 ml of acetone, and it is stirred for 24 hours at room temperature. It is diluted with 300 ml of ethyl acetate and washed once each with 50 ml of water and 50 ml of saturated sodium chloride solution. After drying on sodium sulfate and after filtration, it is concentrated by evaporation in a vacuum. The thus obtained residue is purified by medium-pressure chromatography on silica gel with hexane/ethyl acetate in a 7:3 ratio. In this way, 670 mg of the title compound is obtained.

NMR (300 MHz, DMSO-d6): δ=1.27 (9H), 1.32 (3H), 3.14 (3H), 4.34 (2H), 6.92 (1H), 7.08 (1H), 7.39 (1H), 7.41 (2H), 7.56 (2H), 12.33 (1H).

1e) 5-[(4-tert-Butyl-phenylsulfonyl)-methyl-amino]-3-phenyl-1H-indole-2-carboxylic acid ethyl ester

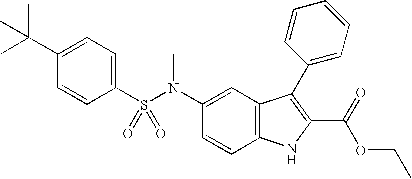

239 mg of phenylboronic acid and 3.37 ml of a 1M aqueous sodium carbonate solution as well as 160 mg of lithium chloride are added to a solution of 666 mg of the ester, produced in Example 1d), in a mixture that consists of 25.5 ml of ethanol and 25.5 ml of toluene. After 125 mg of tetrakis(triphenylphosphine)-palladium is added, the reaction mixture is refluxed for 20 hours. After cooling to room temperature, it is suctioned off on Celite and rewashed with ethyl acetate. The thus obtained organic phase is washed with 10 ml of saturated sodium bicarbonate solution and saturated sodium chloride solution and dried on sodium sulfate. After concentration by evaporation in a vacuum, the thus obtained residue is purified by chromatography on silica gel with hexane/0-60% ethyl acetate. In this way, 626 mg of the title compound is obtained.

NMR (300 MHz, DMSO-d6): δ=1.13 (3H), 1.27 (9H), 3.06 (3H), 4.18 (2H), 6.90 (1H), 7.07 (1H), 7.25-7.36 (5H), 7.39 (2H), 7.43 (1H), 7.55 (2H), 10.24 (1H).

1f) 5-[(4-tert-Butyl-phenylsulfonyl)-methyl-amino]-3-phenyl-1H-indole-2-carboxylic acid

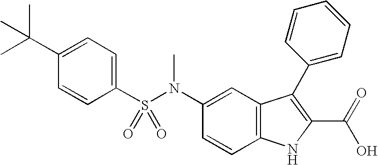

905 mg of sodium hydroxide is added to a mixture of 600 mg of the ester, produced in Example 1e), in 14.5 ml of ethanol and 7.25 ml of ethanol, and it is stirred for 20 hours at room temperature. Then, it is diluted with 100 ml of water and acidified with 5% sulfuric acid. The deposited precipitate is filtered off and dried. In this way, 205 mg of the title compound, which is further reacted without additional purification, is obtained.

NMR (300 MHz, DMSO-d6): δ=1.26 (9H), 3.06 (3H), 6.88 (1H), 7.04 (1H), 7.24-7.35 (5H), 7.36-7.43 (3H), 7.55 (2H), 11.94 (1H), 12.93 (1H).

### EXAMPLE 2

### 5-[(4-tert-Butyl-phenylsulfonyl)-methyl-amino]-3-phenyl-1H-indole-2-carboxylic acid (2-morpholin-4-yl-ethyl)-amide

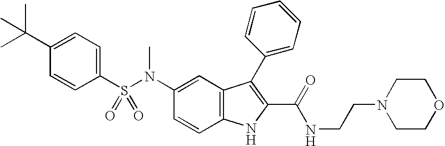

Analogously to Example 1, 36.2 mg of the title compound is obtained from 45 mg of the acid of Example 1f) and 12.8 μl of 2-(morpholin-4-yl)-ethylamine.

NMR (300 MHz, DMSO-d6): δ=1.27 (9H), 2.18 (4H), 2.25 (2H), 3.06 (3H), 3.25 (2H), 3.38 (4H), 6.85 (1H), 6.98 (1H), 7.27-7.42 (9H), 7.55 (2H), 11.85 (1H).

### EXAMPLE 3

### (±)-5-[(4-tert.-Butyl-phenylsulfonyl)-methyl-amino]-3-phenyl-1H-indole-2-carboxylic acid-(2-hydroxy-1-methyl-ethyl)-amide

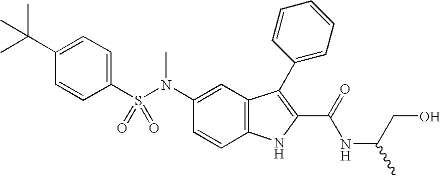

Analogously to Example 1, 42.6 mg of the title compound is obtained from 45 mg of the acid of Example 1f) and 7.75 μl of 2-amino-1-propanol.

NMR (300 MHz, DMSO-d6): δ=0.94 (3H), 1.26 (9H), 3.07 (3H), 3.13-3.30 (2H), 3.89 (1H), 4.62 (1H), 6.89 (1H), 6.96 (1H), 6.99 (1H), 7.26-7.41 (8H), 7.54 (2H), 11.83 (1H).

### EXAMPLE 4

### (±)-5-[(4-tert-Butyl-phenylsulfonyl)-methyl-amino]-3-phenyl-1H-indole-2-carboxylic acid-(2-hydroxy-propyl)-amide

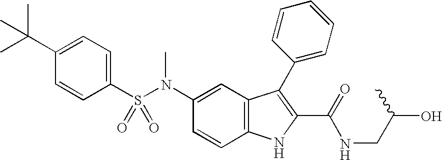

Analogously to Example 1, 45.4 mg of the title compound is obtained from 45 mg of the acid of Example 1f) and 7.31 mg of 1-amino-2-propanol.

NMR (300 MHz, DMSO-d6): δ=0.91 (3H), 1.27 (9H), 3.00-3.17 (2H), 3.06 (3H), 3.58 (1H), 4.59 (1H), 6.87 (1H), 6.99 (1H), 7.15 (1H), 7.26-7.41 (8H), 7.54 (2H), 11.84 (1H).

### BIOLOGICAL EXAMPLES

### Example 1

### sAC-Assay

In a suitable buffer system, the soluble, sperm-specific adenylate cyclase catalyzes the reaction of adenosine triphosphate (ATP) to cyclic adenosine monophosphate (cAMP) and pyrophosphate. Free cAMP that is generated in this way is then used in a competitive detection process, in which the binding of a europium kryptate (Eu[K])-labeled anti-cAMP antibody (anti cAMP-Eu[K]-AK) to a cAMP-molecule-labeled, modified allophycocyanine-1 molecule (cAMP-XL665) is prevented. In the absence of exogenic cAMP, after excitation at 335 nm, it results in a fluorescence resonance energy transfer (FRET) between the anti cAMP-Eu[K]-AK (FRET-donor) and the cAMP-XL665 molecule (FRET-acceptor). This process is quantified at different times (time-resolved) based on the emission of FRET-acceptor XL665 (665 nm and 620 nm). A signal drop (measured as a wave ratio; calculation formula: [(E665 nm/E620 nm)×10000]) can be attributed to the presence of cAMP and thus to the activity of sAC.

First, 1.5 μl of the test substance (in 30% DMSO), only 30% DMSO in the solvent controls, is introduced per recess in a 384-hole test plate (polystyrene; 384, NV). Then, 10 μl of a dilute sAC enzyme solution is recovered (enzyme stock solution in 300 mmol of NaCl, 10% glycerol; pH 7.6; intermediate and final enzyme dilution a) 1:10 and b) 1:2000 in each case in: 1.0 mmol of MnCl₂; 0.2% BSA; 50 mmol of tris, pH 7.5 in H₂O). The enzyme reaction is started by adding 5 μl of the ATP-substrate solution (200 μmol of ATP in H₂O) and is completed after incubation (25 minutes at room temperature) by the addition of 5 μl of stop solution (200 μmol of EDTA in PBS). Finally, the entire reaction is adjusted to a total volume of 91.5 μl by adding 70 μl of PBS.

Then, 8 μl of detection solution 1 is introduced into a recess of the 384-hole measuring plate (measuring plate: polystyrene; 384, SV—black; detection solution 1: 50 μl of cAMP-XL665; 950 μl of reconstitution buffer; 2200 μl of PBS; cAMP-XL665: production by the addition of 5 ml of H₂0 to the freeze-dried product as specified by Cis Bio Kit: #62AMPPEC instructions; storage: aliquoted at −80° C.). Then, 3 μl from the 91.5 μl is added to the corresponding recess of the test plate. Finally, the addition of 8 μl of detection solution 2 (detection solution 2: 50 μl of anti cAMP-Eu[K]-AK; 950 μl of reconstitution buffer; 2200 μl of PBS; anti cAMP-Eu[K]-AK: production as specified by Cis Bio Kit: #62AMPPEC instructions; storage: aliquoted at −80° C.) is carried out.

After an additional incubation of 90 minutes at room temperature, the HTRF result is measured either on the Packard Discovery or with the RubiStar HTRF measuring device (Delay: 50 μs; Integration time: 400 μs).

### Example 2

### Isolation of Human Sperm from Ejaculates and Capacitation

2.1. Isolation of Sperm

Human sperm are purified from the ejaculate by a two-layer gradient system based on colloidal silica particles (trade name: Percoll or ISolate).

Per ejaculate, 2.5 ml each of a preheated lower layer (“90% ISolate lower layer,” Irvine Company) is introduced into a 15 ml centrifuging tube (conical, plastic) and carefully covered with 2.5 ml of a preheated upper layer (“50% ISolate upper layer,” Irvine Company) and held back in a water bath at 37° C. for <1 hour. The gradient is carefully coated with a maximum of 3 ml of normal (relative to the number of sperm, motility and liquefaction) ejaculate. The sedimentation of sperm is carried out at 1000×g for 25 minutes at room temperature. By means of a glass capillary, both layers are suctioned off to a point just above the sperm pellets. To wash out the ISolate gradients, the sperm pellets that are resuspended in about 200 μl each are moved into a 15 ml plastic tube with 12 ml of mHTF medium (4 mmol of NaHCO₃; 0.01% BSA; 37° C.), and the sperm are sedimented at 1000×g for 20 minutes. The medium is suctioned off to a point just above the pellet and adjusted to 1000 μl with mHTF medium (4 mmol of NaHCO₃; 0.01% BSA; 37° C.). The number of sperm is determined in a Neubauer counting chamber, and adjusted for the following capacitation optionally with mHTF medium (4 mmol of NaHCO₃; 0.01% BSA; 37° C.) to 4×106 sperm/150 μl.

2.2. Capacitation

If the effect of test substances on the acrosomal reaction is to be tested, the sperm must be pre-incubated with the test substances. This pre-incubation (15 minutes in the incubator at 37° C.) is necessary to make possible the penetration of test substances in the sperm before the beginning of capacitation, i.e., to achieve a pre-saturation of the binding sites in the sperm, in particular in substances that do not pass through the membrane well. In addition, it is necessary since the increase of the BSA concentration in the capacitation by the high lipid bond of the BSA could result in the reduction of the effective test substance concentration in the preparation.

The test substances are dissolved in DMSO and diluted with mHTF medium (4 mmol of NaHCO₃; 0.01% BSA; 37° C.), such that in the final capacitation preparation of 400 μl, the DMSO concentration is 0.5%. 150 μl each of the tempered test substance solution above is pipetted in each case into 150 μl of sperm suspension and pre-incubated for 15 minutes at 37° C. The capacitation of sperm is started by adding 100 μl of mHTF-medium (88 mmol of NaHCO₃; 4% BSA; 37° C.). In the final 400 μl of capacitation preparation, the sperm concentration is 10×106/ml, the bicarbonate concentration is 4 mmol, and the BSA concentration is 1%. The capacitation is carried out at 37° C. for 3 hours in an incubator.

To complete the capacitation, the preparations (400 μl each) are each transferred completely into a 15 ml sample tube with 1.5 ml of mHTF (4 mmol of NaHCO₃; 37° C.), centrifuged for 5 minutes at 1000×g, and the supernatant is removed. With this step, both the high amount of protein and the test substances are removed.

### Example 3

### Flow-Cytometric Determination of the Acrosomal Reaction

3.1. Introduction of the Acrosomal Reaction by Ionophore Treatment and Simultaneous CD46-FITC Staining

The acrosomal reaction (AR) of the sperm is triggered by the binding of the sperm to the Zona pellucida (ZP). In this case, enzymes are released from the acrosome that make it possible for the sperm to penetrate the ovocyte through the ZP. In the case of AR, in sperm, it results in a partial merging of the plasma membrane with the outside acrosomal membrane (OAM). The head of the sperm cell is limited only by the inside acrosomal membrane (IAM) at the end. The CD46-antigen can be detected only on the IAM.

The acrosomal reaction can be induced in vitro with a suitable concentration of the calcium-ionophore A23187 on capacitated sperm, but not on uncapacitated sperm or on sperm that are inhibited in capacitation by test substances. With the aid of FITC-labeled anti-CD46 antibodies (Pharmingen Company) against the IAM, the acrosome-reacted sperm can be distinguished in the flow cytometer from the acrosome-intact sperm, in which the IAM is not exposed. By the simultaneous staining of the sperm with the DNA dye ethidium homodimer (EhD), which stains only the DNA membrane-defective, thus dead cells, the dead sperm can be distinguished from the living sperm.

Since the ionophore dilutions seem to be very unstable in triggering the AR and must be mixed for the simultaneous staining with the CD46-FITC solution, the solutions cannot be prepared before the beginning of the test but rather must be produced during the working-up of the capacitation preparations.

The sperm pellets are resuspended in the residual supernatant and are diluted in a water bath (37° C.) with 450 ∥l of mHTF (4 mmol of NaHCO₃; 0.01% BSA; 37° C.). 100 μl Aliquots of the sperm suspensions are pipetted into prepared FACS-flow tube samples (in a water bath). 150 μl of a solution with ionophore and FITC-labeled anti-CD46 antibodies are pipetted into the sperm. The final concentration is 800 nmol of ionophore and a 1:125 dilution of the anti-CD46 antibody in mHTF (4 mmol of NaHCO₃; 0.01% BSA; 37° C.). The sperm are incubated therein, protected from light, for 30 minutes in a water bath at 37° C.

The incubation is stopped by adding 3.5 ml of PBS [0.1% BSA]/preparation, followed by centrifuging for 5 minutes at 700×g (room temperature) and subsequent suctioning-off of the supernatants. After the centrifuging, the samples are kept warm on the hot plate until the measurement is done.

3.2. EhD Staining (for Differentiation of Dead/Living Acrosomally-Reacted Sperm)

After suctioning-off, the sperm pellets are mixed with 500 μl each of freshly prepared EhD solution (150 nmol of EhD in PBS [w/o BSA]; 37° C.). The samples can then be measured in a flow cytometer (BD Facs Calibur). The measurement is done at a laser excitation wavelength of 488 nm; 10,000 sperm per measurement are detected. Acrosome-reacted sperm are measured with CD46-FITC in an FL-1 filter at 530 nm. Dead sperm are measured by means of EhD-DNA-staining in an FL-2 filter at 634 nm. The measuring channels are first compensated appropriately with respect to one another.

3.3 Evaluation

The sperm are selected as a very uniform cell population in an FSC-H (forward scatter) from SSC-H (sideward scatter) Dotblot. Since a two-color fluorescence staining is used, the evaluation is carried out with the aid of a quadrant analysis in an FL-1 (EhD, X-axis) vs. FL-2 (FITC-CD46, Y-axis) Dotblot with the selected sperm population from the FSC vs. SSC Dotblot:

| | Quadrant | | |
|---|---|---|---|
| | in FL-1 | | |
| | vs. FL-2 | | |
| | Dotblot | Staining | Analysis |
| Q1 = UL | Upper left | Only EhD | Dead, non-acrosomally-reacted |
| | | | sperm |
| Q2 = UR | Upper right | EhD and | Dead, acrosomally-reacted |
| | | FITC-CD46 | sperm |
| Q3 = LL | Lower left | Unstained | Living, non-acrosomally-reacted |
| | | | sperm |
| Q4 = LR | Lower right | Only | Living, acrosomally-reacted |
| | | FITC-CD46 | sperm |

To calculate the % of induced, acrosomally-reacted sperm (=“IAR[%]”), only the living sperm from Q3 and Q4 are used, and their total number is set at equal to 100%. IAR is then calculated as follows:

$$IAR [ \% ] = \frac{LR \times 100}{LL + LR}$$

A portion of the sperm already reacts spontaneously acrosomally without the addition of ionophore (=“SAR[%]”). Therefore, a control measurement of identically-treated sperm without the addition of an ionophore is always also taken. The SAR is calculated analogously to the IAR. The acrosomal reaction (=“ARIC[%]”) that is actually triggered by the ionophore is calculated as the difference: ARIC=IAR−SAR.

For the following analysis of the effect of our inhibitors on the sAC-mediated capacitation (measured as the ability of the sperm to undergo ionophore-induced acrosomal reaction), the percentage of acrosomally-reacted sperm in the positive capacitation control (=incubation with mHTF medium with 25 mmol of NaHCO3; 1% BSA without test substances) is set at =100%. The ability of the sperm mixed with the test substances to undergo acrosomal reaction is indicated relative to this maximum acrosomal reaction.

Materials Used

mHTF=modif. human tubular fluid (Irvine Scientific Company), Dulbecco's phosphate-buffered saline (Gibco Company) (with Ca²+, Mg²+, 1 g/l of D-glucose, 36 mg/l of Na-pyruvate, w/o phenol red, w/o NaHCO₃); bovine serum albumin, Fraction V (Fluka Company); dimethyl sulfoxide (DMSO), anhydrous (Merck Company); sodium bicarbonate 7.5% solution (893 mmol) (Irvine Scientific Company); isolate gradient (Irvine Scientific Company); Ionophore-A23187 free acid, (Calbiochem Company); ethidium homodimer (EhD) (Molecular Probe Company), Mouse Anti Human CD46:FITC (Pharmingen Company).

### BIBLIOGRAPHICAL REFERENCES

J. W. Carver-Ward, Human Reproduction Vol. 11, No. 9, pp: 1923 ff, 1996 High Fertilization Prediction by Flow-Cytometric Analysis of the CD46 Antigen on the Inner Acrosomal Membrane of Spermatozoa

O. J. D'Cruz, G. G. Haas, Fertility and Sterility Vol. 65, No. 4, pp: 843 ff, 1996 Fluorescence-Labeled Fucolectins are Superior Markers for Flow-Cytometric Quantitation of the Sperm Acrosome Reaction

E. Nieschlag, H. M. Behre, Andrologie [Andrology], Springer Verlag 1996

### Examples

| | | | IC50 |
|---|---|---|---|
| # | R⁴ | R⁶ | (μM) |
| 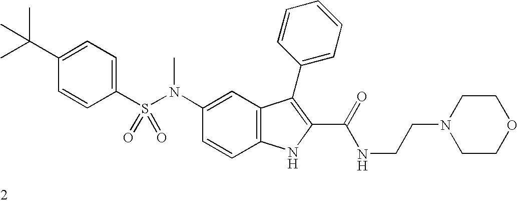 | 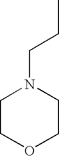 | CH₃ | 2.6 |
| 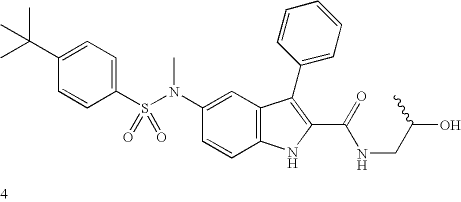 | —CH₂—CHCH₃—OH | CH₃ | 1.3 |
| 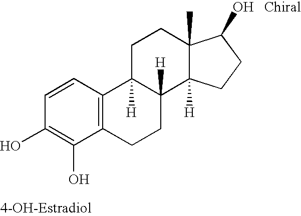 | | | 13 |
| 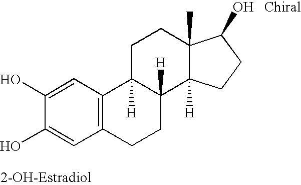 | | | 11 |

It can be seen from the table that the compounds according to the invention relative to the inhibition of the soluble adenylate cyclase, expressed by the IC₅₀ value, sometimes have an approximately 10× higher activity than the already known catechol estrogens (OH-estradiols). The catechol estrogens are toxic, therefore the compounds according to the invention are far superior to the known compounds. The compounds according to the invention are also approximately 10× more powerful than the compounds presented by Zippin.

Without further elaboration, it is believed that one skilled in the art can, using the preceding description, utilize the present invention to its fullest extent. The preceding preferred specific embodiments are, therefore, to be construed as merely illustrative, and not limitative of the remainder of the disclosure in any way whatsoever.

In the foregoing and in the examples, all temperatures are set forth uncorrected in degrees Celsius and, all parts and percentages are by weight, unless otherwise indicated.

The entire disclosures of all applications, patents and publications, cited herein and of corresponding German application No. 10 2006 014 320.5, filed Mar. 23, 2006, and U.S. Provisional Application Ser. No. 60/788,076 filed Apr. 3, 2006, are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Compounds of general formula (I), 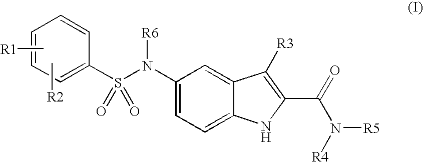
whereby
R¹ means hydrogen, halogen, CF₃, C₃-C₆-cycloalkyl, which optionally is polysaturated and optionally is polysubstituted, or the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl optionally can be interrupted in one or more places, in the same way or differently, by oxygen, sulfur or nitrogen, or the group sulfonyl-C₁-C₆-alkyl, sulfonamide, or cyano,
R² means halogen, CF₃, C₃-C₆-cycloalkyl, which optionally is polysaturated and optionally is polysubstituted, or the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl optionally can be interrupted in one or more places, in the same way or differently, by oxygen, sulfur or nitrogen, or the group sulfonyl-C₁-C₆-alkyl, sulfonamide, or cyano,
R³ means C₆-C₁₂-aryl, which optionally can be substituted in
one or more places, in the same way or differently, with halogen, with C₁-C₆-alkyl or C₁-C₆-acyl, which optionally can be substituted in one or more places, or can be substituted with C₁-C₆-alkoxy, hydroxy, cyano, CO₂-(C₁-C₆-alkyl), N-(C₁-C₆-alkyl)₂, CO—NR⁴R⁵ or with CF₃;
C₅-C₁₂-heteroaryl, which optionally can be substituted in one or more places, in the same way or differently, with halogen, C₁-C₆-alkyl, C₁-C₆-acyl, C₁-C₆-alkoxy, hydroxy, cyano, CO₂-(C₁-C₆-alkyl), N-(C₁-C₆-alkyl)_{2,} CO—NR⁴R⁵ or with CF₃; or C₃-C₆-cycloalkyl, which optionally can be substituted in one or more places, in the same way or differently, with halogen, CF₃, hydroxy, cyano, CO₂-(C₁-C₆-alkyl), C₁-C₆-alkyl, C₁-C₆-acyl, N-(C₁-C₆-alkyl)₂, CO—NR⁴R⁵ or C₁-C₆-alkoxy,
R⁴ means hydrogen, C₃-C₆-cycloalkyl, which optionally is substituted in
one or more places, in the same way or differently, with C₁-C₆-alkyl, C₁-C₆-acyl, C₁-C₆-alkoxy or CF₃;
C₆-C₁₂-aryl, which optionally is substituted in one or more places, in the same way or differently, with halogen, C₁-C₆-alkyl, C₁-C₆-acyl, C₁-C₆-alkoxy, N-C₁-C₆-alkyl-C₁-C₆-alkyl, CF₃ or cyano; or
C₅-C₁₂-heteroaryl, which optionally is substituted in one or more places, in the same way or differently, with halogen, C₁-C₆-alkyl, C₁-C₆-acyl, C₁-C₆-alkoxy, N-C₁-C₆-alkyl-C₁-C₆-alkyl, CF₃ or cyano; or
C₁-C₆-alkyl, which can be substituted in any way desired,
R⁵ means hydrogen, C₁-C₆-alkyl-C₃-C₆-cycloalkyl, which optionally is substituted in one or more places, in the same way or differently, with C₁-C₆-alkyl, C₁-C₆-acyl, C₁-C₆-alkoxy or CF₃;
C₃-C₆-cycloalkyl, which optionally is substituted in one or more places, in the same way or differently, with C₁-C₆-alkyl, C₁-C₆-acyl, C₁-C₆-alkoxy or CF₃; C₆-C₁₂-aryl, which optionally is substituted in one or more places, in the same way or differently, with halogen, C₁-C₆-alkyl, C₁-C₆-acyl, C₁-C₆-alkoxy, N-C₁-C₆-alkyl-C₁-C₆-alkyl, CF₃ or cyano; or
C₅-C₁₂-heteroaryl, which optionally is substituted in one or more places, in the same way or differently, with halogen, C₁-C₆-alkyl, C₁-C₆-acyl, C₁-C₆-alkoxy, N-C₁-C₆-alkyl-C₁-C₆-alkyl, CF₃ or cyano; or
C₁-C₆-alkyl, which can be substituted in any way desired,
R⁴ and R⁵ together form a 5- to 8-membered ring, which can contain additional heteroatoms, and
R⁶ means the group C₁-C₆-alkyl, C₁-C₆-acyl, C₁-C₆-alkyl-cyclo-C₃-C₆-alkyl, C₁-C₆-alkyl-C₆-C₁₂-aryl, in which C₁-C₆-alkyl, C₁-C₆-acyl, C₁-C₆-alkyl-cyclo-C₃-C₆-alkyl, or C₁-C₆-alkyl-C₆-C₁₂-aryl optionally can be substituted in one or more places, in the same way or differently, by hydroxy, methoxy, ethoxy, iso-propoxy, chlorine, bromine, fluorine, cyano, methyl-sulfonyl or amino-sulfonyl,
as well as the isomers, diastereomers, enantiomers and salts thereof.

2. Compounds according to claim 1, whereby
R¹ means hydrogen, halogen, CF₃, C₃-C₆-cycloalkyl, or the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl optionally can be interrupted in one or more places, in the same way or differently, by oxygen, sulfur or nitrogen, or the group sulfonyl-C₁-C₆-alkyl, sulfonamide, or cyano,
R² means halogen, CF₃, or C₃-C₆-cycloalkyl, or the group C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl, C₁-C₆-aryl-C₁-C₆-alkyl or CF₃, in which C₁-C₆-alkyl, C₁-C₆-aryl, C₁-C₆-acyl, halo-C₁-C₆-alkyl, C₁-C₆-alkyl-C₁-C₆-alkyl, C₁-C₆-alkyl-C₁-C₆-acyl, C₁-C₆-acyl-C₁-C₆-acyl, C₁-C₆-alkyl-C₁-C₆-aryl or C₁-C₆-aryl-C₁-C₆-alkyl optionally can be interrupted in one or more places, in the same way or differently, by oxygen, sulfur or nitrogen, or the group sulfonyl-C₁-C₆-alkyl, sulfonamide, or cyano,
R³ means C₆-C₁₂-aryl, which optionally can be substituted in
one or more places, in the same way or differently, with halogen, with C₁-C₆-alkyl, C₁-C₃-acyl, C₁-C₃-alkoxy, cyano, hydroxy, N—(CH₃)₂, CO₂-(C₁-C₃-alkyl), CO—NR⁴R⁵ or CF₃;
C₅-C₁₂-heteroaryl, which optionally can be substituted in one or more places, in the same way or differently, with chlorine and/or fluorine, with C₁-C₆-alkyl, C₁-C₃-acyl, C₁-C₃-alkoxy, cyano, hydroxy, N—(CH₃)₂, CO₂-(C₁-C₃-alkyl), CO—NR⁴R⁵ or with CF₃;
C₃-C₆-cycloalkyl, which optionally can be substituted in one or more places, in the same way or differently, with chlorine and/or fluorine, CF₃, cyano, C₁-C₃-alkyl, C₁-C₃-acyl, hydroxy, N—(CH₃)₂, CO₂-(C₁-C₃-alkyl), CO—NR⁴R⁵ or C₁-C₃-alkoxy,
R⁴ means hydrogen, C₃-C₆-cycloalkyl, which optionally is substituted in one or more places, in the same way or differently, with C₁-C₃-alkyl, C₁-C₃-acyl, C₁-C₃-alkoxy or CF₃;
C₆-C₁₂-aryl, which optionally is substituted in one or more places, in the same way or differently, with halogen, C₁-C₃-alkyl, C₁-C₃-acyl, C₁-C₃-alkoxy, N-C₁-C₃-alkyl-C₁-C₃-alkyl, CF₃ or cyano; or
C₅-C₁₂-heteroaryl, which optionally is substituted in one or more places, in the same way or differently, with halogen, with C₁-C₃-alkyl, C₁-C₃-acyl, C₁-C₃-alkoxy, N-C₁-C₃-alkyl-C₁-C₃-alkyl, CF₃ or cyano; or
C₁-C₆-alkyl, which can be substituted in any way desired,
R⁵ means hydrogen, C₁-C₆-alkyl-C₃-C₆-cycloalkyl, which optionally is substituted in one or more places, in the same way or differently, with C₁-C₆-alkyl, C₁-C₆-acyl, C₁-C₆-alkoxy or CF₃;
C₃-C₆-cycloalkyl, which optionally is substituted in one or more places, in the same way or differently, with C₁-C₃-alkyl, C₁-C₃-acyl, C₁-C₃-alkoxy or CF₃;
C₆-C₁₂-aryl, which optionally is substituted in one or more places, in the same way or differently, with halogen, C₁-C₃-alkyl, C₁-C₃-acyl, C₁-C₃-alkoxy, N-C₁-C₃-alkyl-C₁-C₃-alkyl, CF₃ or cyano; or
C₅-C₁₂-heteroaryl, which optionally is substituted in one or more places, in the same way or differently, with halogen, with C₁-C₃-alkyl, C₁-C₃-acyl, C₁-C₃-alkoxy, N-C₁-C₃-alkyl-C₁-C₃-alkyl, CF₃ or cyano; or
C₁-C₆-alkyl, which can be substituted in any way desired,
R⁴ and R⁵ together form a 5- to 8-membered ring, which can contain additional heteroatoms, and
R⁶ means the group C₁-C₆-alkyl, C₁-C₆-alkyl-cyclo-C₃-C₆-alkyl, C₁-C₆-alkyl-C₆-C₁₂-aryl, in which C₁-C₆-alkyl, C₁-C₆-alkyl-cyclo-C₃-C₆-alkyl, or C₁-C₆-alkyl-C₆-C₁₂-aryl optionally can be substituted in one or three places, in the same way or differently, by hydroxy, methoxy, ethoxy, iso-propoxy, chlorine, bromine, fluorine, cyano, methyl-sulfonyl or amino-sulfonyl,
as well as the isomers, diastereomers, enantiomers and salts thereof.

3. Compounds according to claim 1, whereby
R¹ means hydrogen,
R² means C₃-C₆-cycloalkyl, C₁-C₆-alkyl, CF₃, cyano, bromine, or the group —OCF₃, or —SO₂—CH₃,
R³ means C₆-C₁₂-aryl, which optionally can be substituted in one or more places, in the same way or differently, with halogen, C₁-C₆-alkyl, C₁-C₃-acyl, C₁-C₃-alkoxy, cyano, hydroxy, N—(CH₃)₂, CO₂-(C₁-C₃-alkyl), CO—NR⁴R⁵ or CF₃;
C₅-C₁₂-heteroaryl, which optionally can be substituted in one or more places, in the same way or differently, with chlorine and/or fluorine, with C₁-C₆-alkyl, C₁-C₃-acyl, C₁-C₃-alkoxy, cyano, hydroxy, N—(CH₃)₂, CO₂-(C₁-C₃-alkyl), CO—NR⁴R⁵ or with CF₃;
C₃-C₆-cycloalkyl, which optionally can be substituted in one or more places, in the same way or differently, with chlorine and/or fluorine, CF₃, cyano, C₁-C₃-alkyl, C₁-C₃-acyl, hydroxy, N—(CH₃)₂, CO₂-(C₁-C₃-alkyl), CO—NR⁴R⁵ or C₁-C₃-alkoxy,
R⁴ means hydrogen,
R⁵ means hydrogen, C₁-C₆-alkyl-C₃-C₆-cycloalkyl, which optionally is substituted in one or more places, in the same way or differently, with C₁-C₆-alkyl, C₁-C₆-acyl, C₁-C₆-alkoxy or CF₃;
C₃-C₆-cycloalkyl, which optionally is substituted in one or more places, in the same way or differently, with C₁-C₃-alkyl, C₁-C₃-acyl, C₁-C₃-alkoxy or CF₃;
C₆-C₁₂-aryl, which optionally is substituted in one or more places, in the same way or differently, with halogen, C₁-C₃-alkyl, C₁-C₃-acyl, C₁-C₃-alkoxy, N-C₁-C₃-alkyl-C₁-C₃-alkyl, CF₃ or cyano; or
C₅-C₁₂-heteroaryl, which optionally is substituted in one or more places, in the same way or differently, with halogen, C₁-C₃-alkyl, C₁-C₃-acyl, C₁-C₃-alkoxy, N-C₁-C₃-alkyl-C₁-C₃-alkyl, CF₃ or cyano;
or C₁-C₆-alkyl, which can be substituted in any way desired,
R⁶ means the group C₁-C₄-alkyl, CH₂-cyclo-C₃-C₆-alkyl, CH₂-C₆-C₁₂-aryl, in which C₁-C₄-alkyl, CH₂-cyclo-C₃-C₆-alkyl, or CH₂-C₆-C₁₂-aryl optionally can be substituted in one or three places, in the same way or differently, by hydroxy, methoxy, chlorine, fluorine, cyano or amino-sulfonyl,
as well as the isomers, diastereomers, enantiomers and salts thereof.

4. Compounds according to claim 1, whereby
R¹means hydrogen,
R² means C₃-C₆-cycloalkyl, C₁-C₆-alkyl, CF₃, cyano, bromine, or the group —OCF₃, or —SO₂—CH₃ and is in para-position,
R³ means C₆-C₁₂-aryl, which optionally can be substituted in one or two places, in the same way or differently, with halogen, C₁-C₃-alkyl, acetyl, methoxy, ethoxy, cyano, hydroxy, N—(CH₃)₂, CO₂-(C₁-C₃-alkyl), CO—NHR⁵ or CF₃;
C₅-C₁₂-heteroaryl, which optionally can be substituted in one or two places, in the same way or differently, with chlorine and/or fluorine, with C₁-C₃-alkyl, acetyl, methoxy, ethoxy, cyano, hydroxy, N—(CH₃)₂, CO₂-(C₁-C₃-alkyl), CO—NHR⁵ or with CF₃; or C₃-C₆-cycloalkyl,
R⁴ means hydrogen,
R⁵ means hydrogen, or C₁-C₆-alkyl-C₃-C₆-cycloalkyl, which optionally is substituted in one or more places, in the same way or differently, with C₁-C₆-alkyl, C₁-C₆-acyl, C₁-C₆-alkoxy or CF₃;
C₃-C₆-cycloalkyl, which optionally is substituted in one or more places, in the same way or differently, with C₁-C₃-alkyl, C₁-C₃-acyl, C₁-C₃-alkoxy or CF₃;
C₆-C₁₂-aryl, which optionally is substituted in one or more places, in the same way or differently, with halogen, C₁-C₃-alkyl, C₁-C₃-acyl, C₁-C₃-alkoxy, N-C₁-C₃-alkyl-C₁-C₃-alkyl, CF₃ or cyano; or
C₅-C₁₂-heteroaryl, which optionally is substituted in one or more places, in the same way or differently, with halogen, C₁-C₃-alkyl, C₁-C₃-acyl, C₁-C₃-alkoxy, N-C₁-C₃-alkyl-C₁-C₃-alkyl, CF₃ or cyano; or
C₁-C₆-alkyl, which can be substituted in any way desired,
R⁶ means the group C₁-C₄-alkyl, CH₂-cyclo-C₃-C₆-alkyl, CH₂-C₆-C₁₂-aryl, in which C₁-C₄-alkyl, CH₂-cyclo-C₃-C₆-alkyl, or CH₂-C₆-C₁₂-aryl optionally can be substituted in one or three places, in the same way or differently, by hydroxy, methoxy, chlorine, fluorine, cyano or amino-sulfonyl,
as well as the isomers, diastereomers, enantiomers and salts thereof.

5. Compounds according to claim 1, whereby
R¹ means hydrogen,
R² means tert-butyl, iso-propyl, iso-butyl, sec-butyl, cyano, bromine, or the group —O—CF₃, or —SO₂—CH₃ and is in para-position,
R³ means the group 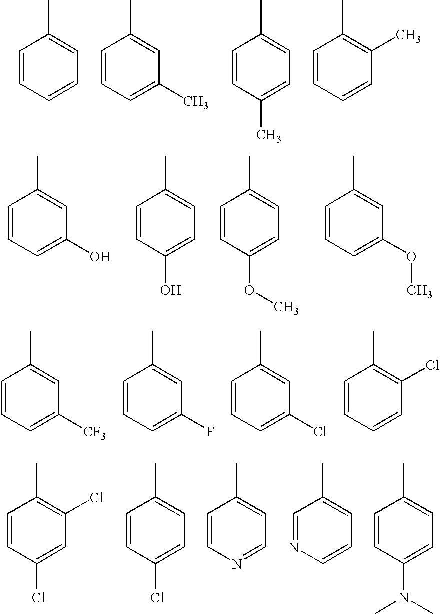
R⁴ means hydrogen,
R⁵ means hydrogen or the group —CH₂)ₙ—N—(CH₃)₂, —(CH₂)₂—CH₃, —CH₂)₂—NH—COCH₃, —CH₂)—CHCH₃—OH, —CH₂)₂—O—CH₃, —(CH₂)₂—OH, or —CHCH₃—CH₂—OH, whereby m=1-3, 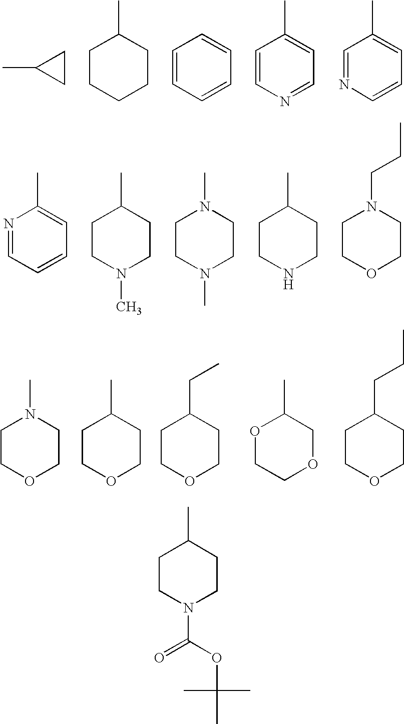
R⁶ means methyl, ethyl, propyl, 2-methoxy-ethyl, —CH₂—CF₃, —(CH₂)₂—CF₃ and benzyl,
as well as the isomers, diastereomers, enantiomers and salts thereof.

6. Compounds according to claim 1, whereby
R¹ means hydrogen,
R² means tert-butyl, iso-propyl, iso-butyl, sec-butyl, cyano, bromine, or the group —O—CF₃, or —SO₂—CH₃ and is in para-position,
R³ means the group 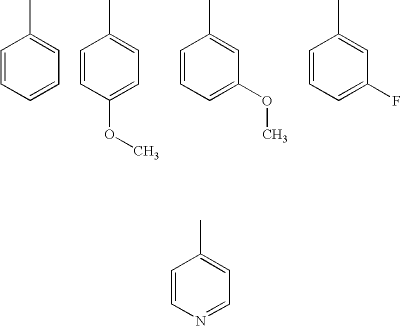
R⁴ means hydrogen,
R⁵ means hydrogen or the group —CH₂)—CHCH₃—OH, —(CH₂)₂—O—CH₃, or —CHCH₃—CH₂—OH, 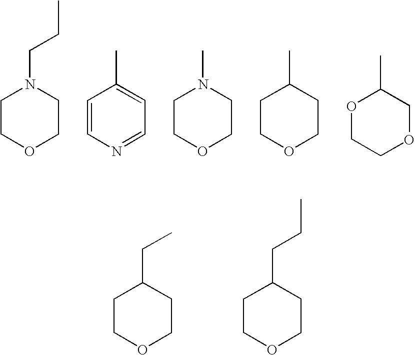
R⁶ means methyl, ethyl, propyl, 2-methoxy-ethyl, —CH₂—CF₃, —(CH₂)₂—CF₃ and benzyl.

7. Compounds according to claim 1, selected from a group that contains the following compounds:
5-[(4-tert-Butyl-phenylsulfonyl)-methyl-amino]-3-phenyl-1H-indole-2-carboxylic acid-(tetrahydro-pyran-4-yl)-amide
2. 5-[(4-tert-Butyl-phenylsulfonyl)-methyl-amino]-3-phenyl-1H-indole-2-carboxylic acid (2-morpholin-4-yl-ethyl)-amide
3. (±)-5-[(4-tert-Butyl-phenylsulfonyl)-methyl-amino]-3-phenyl-1H-indole-2-carboxylic acid-(2-hydroxy-1-methyl-ethyl)-amide
4. (±)-5-[(4-tert-Butyl-phenylsulfonyl)-methyl-amino]-3-phenyl-1H-indole-2-carboxylic acid-(2-hydroxy-propyl)-amide
5. 5-[(4-tert-Butyl-phenylsulfonyl)-methyl-amino]-3-phenyl-1H-indole-2-carboxylic acid-(pyridin-4-yl)-amide
6. 5-[(4-tert-Butyl-phenylsulfonyl)-benzyl-amino]-3-phenyl-1H-indole-2-carboxylic acid-(tetrahydro-pyran-4-yl)-amide
7. 5-[(4-tert-Butyl-phenylsulfonyl)-benzyl-amino]-3-phenyl-1H-indole-2-carboxylic acid (2-morpholin-4-yl-ethyl)-amide
8. (±)-5-[(4-tert-Butyl-phenylsulfonyl)-benzyl-amino]-3-phenyl-1H-indole-2-carboxylic acid-(2-hydroxy-1-methyl-ethyl)-amide
9. (±)-5-[(4-tert-Butyl-phenylsulfonyl)-benzyl-amino]-3-phenyl-1H-indole-2-carboxylic acid-(2-hydroxy-propyl)-amide
10. 5-[(4-tert-Butyl-phenylsulfonyl)-(2-methoxy-ethyl)-amino]-3-phenyl-1H-indole-2-carboxylic acid-(tetrahydro-pyran-4-yl)-amide
11. 5-[(4-tert-Butyl-phenylsulfonyl)-(2-methoxy-ethyl)-amino]-3-phenyl-1H-indole-2-carboxylic acid (2-morpholin-4-yl-ethyl)-amide
12. (±)-5-[(4-tert-Butyl-phenylsulfonyl)-(2-methoxy-ethyl)-amino]-3-phenyl-1H-indole-2-carboxylic acid-(2-hydroxy-1-methyl-ethyl)-amide
13. (±)-5-[(4-tert-Butyl-phenylsulfonyl)-(2-methoxy-ethyl)-amino]-3-phenyl-1H-indole-2-carboxylic acid-(2-hydroxy-propyl)-amide
14. 5-[(4-tert-Butyl-phenylsulfonyl)-methyl-amino]-3-(3-fluoro-phenyl)-1H-indole-2-carboxylic acid-(tetrahydro-pyran-4-yl)-amide
15. 5-[(4-tert-Butyl-phenylsulfonyl)-methyl-amino]-3-(3-fluoro-phenyl)-1H-indole-2-carboxylic acid (2-morpholin-4-yl-ethyl)-amide
16. 5-[(4-tert-Butyl-phenylsulfonyl)-methyl-amino]-3-(3-methoxy-phenyl)-1H-indole-2-carboxylic acid-(tetrahydro-pyran-4-yl)-amide
17. 5-[(4-tert-Butyl-phenylsulfonyl)-methyl-amino]-3-(3-methoxy-phenyl)-1H-indole-2-carboxylic acid (2-morpholin-4-yl-ethyl)-amide

8. Pharmaceutical agents that contain at least one of the compounds according to claim 1.

9. Pharmaceutical agents according to claim 8 in which the compound of general formula 1 is contained in an effective dose.

10. Compounds of general formula I according to claim 1 for the production of pharmaceutical agents.

11. Pharmaceutical agents according to claim 10 for the treatment of diseases.

12. Pharmaceutical agents according to claim 11, whereby the diseases are caused by disorders in cAMP metabolism.

13. Pharmaceutical agents according to claim 10 for contraception.

14. Pharmaceutical agents according to claim 10 for inhibition of soluble adenylate cyclase.

15. Pharmaceutical agents according to claim 10 with suitable formulation substances and vehicles.

16. Use of the compounds of general formula 1 according to claim 1 in the form of a pharmaceutical preparation for enteral, parenteral, vaginal and oral administration.

17. Process for the compounds of general formula (I) whereby the process contains the reaction of the compounds of general formula (II) to form the compounds of general formula (I).

18. Intermediate products of general formulas (II), (VI), (VII), (IX) and (XI).

19. A method of inhibiting adenylate cyclase comprising administering a compound of claim 1.
